(19) United States
(12) Patent Application Publication
(10) Pub. No.: US 2010/0016256 A1
(43) Pub. Date: Jan. 21, 2010

(54) Taxane Compounds for Treating Eye Disease

(76) Inventors: John R. Gebhard, Salt Lake City, UT (US); Dinesh Patel, Salt Lake City, UT (US)

Correspondence Address: THORPE NORTH & WESTERN, LLP., P.O. Box 1219, SANDY, UT, 84091-1219, US

(21) Appl. No.: 12/506,780

(22) Filed: Jul. 21, 2009

Related U.S. Application Data
(60) Provisional application No. 61/135,566, filed on Jul. 21, 2008.

(51) Int. Cl. A61K 31/714; A61P 27/02; A61K 31/337

(52) U.S. Cl. 514/52; 514/449

(57) ABSTRACT

The present invention is directed to methods of treating eye disease. In one embodiment, the method can comprise administering a taxane-cobalamin bioconjugate or another taxane compound to a subject to treat the eye disease. In one embodiment, the bioconjugate can be dissolved in an aqueous solution prior to administration.

The present application claims the benefit of U.S. Provisional Application No. 61/135,566, filed Jul. 21, 2008, which is incorporated herein by reference.

### BACKGROUND

The efficacy of certain drugs in treating disease is often dependent on their toxicity, biological availability, or how readily an effective amount of the drug can be delivered to a specific location in a subject's body, particularly to a specific type of tissue or population of cells. Therefore, methods and compositions that lower toxicity, increase bioavailability, or facilitate drug targeting can be of considerable value to the pharmaceutical and medicinal arts. One approach to this need involves using molecules that have generally understood transport mechanisms and which can be induced to release drugs in site-specific fashion. Another approach to increasing bioavailability can involve using molecules that broaden the options for formulating drugs, so that the drugs can be administered in more effective dosage forms.

One such mechanism involves the use of cobalamin (Cbl). Cobalamin is an essential biomolecule, the size of which prevents it from being taken up from the intestine and into cells by simple diffusion, but rather by facultative transport. Cobalamin must bind to a specific protein, and the resulting complex is actively taken up through a receptor-mediated transport mechanism. In the small intestine, cobalamin binds to intrinsic factor (IF) secreted by the gastric lining. The Cbl-IF complex binds to IF receptors on the lumenal surface of cells in the ileum and is transcytosed across these cells into the bloodstream. Once there, cobalamin binds to one of three transcobalamins (TCs) to facilitate its uptake by cells. The receptor-mediated nature of cobalamin uptake imparts a degree of cell-specificity to cobalamin metabolism, in that cobalamin can be absorbed and metabolized by cells that present the correct receptor(s).

Several patents have utilized cobalamin for various purposes. For example, Grissom et al. has obtained several U.S. Pat. Nos. 6,790,827; 6,777,237; and 6,776,976; using organocobalt complexes. Russell-Jones et al. has also utilized cobalamin to increase uptake of active agents, as described in a series of patents, including U.S. Pat. Nos. 5,863,900; 6,159,502; and 5,449,720. In addition to this, research and development for methods and compositions having increased bioavailability of various pharmaceutical agents continue to be sought.

### SUMMARY

It has been recognized that it would be advantageous to develop compositions and methods for delivery of taxanes. Briefly, and in general terms, the invention is directed to methods of treating an eye disease by administering a taxane covalently bonded to a cobalamin as a cobalamin-taxane bioconjugate to a subject. Alternatively, the method can comprise administering a taxane compound to a subject to treat the eye disease, wherein the taxane compound has a water solubility of at least 50 mg/mi. In one embodiment, paclitaxel is covalently bonded to the cobalt atom of hydroxocobalamin, or more generally, one of the various forms of vitamin B₁₂. In another embodiment, a cobalamin-taxane bioconjugate can be present in an aqueous solution, and can have a water solubility of at least 50 mg/ml, or even over 100 mg/mi. Methods of administering and/or treating an eye disease include administering a cobalamin-taxane conjugate as an intra-ocular, oral, parenteral, or dermal composition.

Additional features and advantages of the invention will be apparent from the detailed description which follows, taken in conjunction with the accompanying drawings, which together illustrate, by way of example, features of the invention.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 is a graph of various treatments after choroidal neovascularization by laser burns of the eye at intervals of 7, 14, and 21 days; and

FIG. 2 is a bar graph of the mean lesion size (μm³) corresponding to various treatments after choroidal neovascularization by laser burns to the eye after 21 days.

Reference will now be made to the exemplary embodiments illustrated, and specific language will be used herein to describe the same. It will nevertheless be understood that no limitation of the scope of the invention is thereby intended.

### DETAILED DESCRIPTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

In describing and claiming the present invention, the following terminology will be used in accordance with the definitions set forth below.

It must be noted that, as used in this specification and the appended claims, the singular forms “a,” “an,” and, “the” include plural referents unless the context clearly dictates otherwise. Thus, for example, reference to “a taxane” can include one or more of such taxanes, and reference to “the cobalamin” can include reference to one or more cobalamins.

As used herein, the terms “formulation” and “composition” can be used interchangeably and refer to at least one pharmaceutically active agent, such as a taxane covalently bonded to the cobalt atom of a cobalamin with a covalent linkage.

The terms “drug,” “active agent,” “bioactive agent,” “pharmaceutically active agent,” and “pharmaceutical,” can also be used interchangeably to refer to an agent or compound that has measurable specified or selected physiological activity when administered to a subject in an effective amount. As used herein, “carrier” or “inert carrier” refers to typical compounds or compositions used to carry drugs, such as polymeric carriers, liquid carriers, or other carrier vehicles with which a bioactive agent may be combined to achieve a specific dosage form. As a general principle, carriers do not substantially react with the bioactive agent in a manner that substantially degrades or otherwise adversely affects the bioactive agent or its therapeutic potential.

As used herein, “administration,” and “administering” refer to the manner in which a drug, formulation, or composition is introduced into the body of a subject. Various art-known routes such as intra-ocular, oral, parenteral, topical, transdermal, and transmucosal can be used for administration. Thus, an intra-ocular administration can be achieved by dissolving a bioconjugate in water and delivering directly to the eye; e.g. via injection, eye drops, gels, or other topicals.

An oral administration can be achieved by swallowing, chewing, dissolution via adsorption to a solid medium that can be delivered orally, or sucking an oral dosage form comprising active agent(s).

Parenteral administration can be achieved by injecting a drug composition intravenously, intra-arterially, intramuscularly, intrathecally, or subcutaneously, etc. Topical administration may involve applying directly to affected tissue, such as directly to the eye. Transdermal administration can be accomplished by applying, pasting, rolling, attaching, pouring, pressing, rubbing, etc., of a transdermal preparation onto a skin surface. Transmucosal administration may be accomplished by bringing the composition into contact with any accessible mucous membrane for an amount of time sufficient to allow absorption of a therapeutically effective amount of the composition. Examples of transmucosal administration include inserting a suppository into the rectum or vagina; placing a composition on the oral mucosa, such as inside the cheek, on the tongue, or under the tongue; or inhaling a vapor, mist, or aerosol into the nasal passage. These and additional methods of administration are well known in the art.

The term “effective amount,” refers to an amount of an ingredient which, when included in a composition, is sufficient to achieve an intended compositional or physiological effect. Thus, a “therapeutically effective amount” refers to a non-lethal amount of an active agent sufficient to achieve therapeutic results in treating a condition for which the active agent is known or taught herein to be effective. Various biological factors may affect the ability of a substance to perform its intended task. Therefore, an “effective amount” or a “therapeutically effective amount” may be dependent on such biological factors. Further, while the achievement of therapeutic effects may be measured by a physician or other qualified medical personnel using evaluations known in the art, it is recognized that individual variation and response to treatments may make the achievement of therapeutic effects a subjective decision. In some instances, a “therapeutically effective amount” of a drug can achieve a therapeutic effect that is measurable by the subject receiving the drug. For example, in metronomic dosing, “the “therapeutic effective amount” may increase or decrease during the therapeutic treatment due to inherent genetic variation. The determination of an effective amount is well within the ordinary skill in the art of pharmaceutical, medicinal, and health sciences.

As used herein, “treat,” “treatment,” or “treating” refers to the process or result of giving medical aid to a subject, where the medical aid can counteract a malady, a symptom thereof, or other related adverse physiological manifestation. Additionally, these terms can refer to the administration or application of remedies to a patient or for a disease or injury; such as a medicine or a therapy. Accordingly, the substance or remedy so applied, such as the process of providing procedures or applications, are intended to relieve illness or injury. As used herein, “reduce” or “reducing” refers to the process of decreasing, diminishing, or lessening, as in extent, amount, or degree of that which is reduced. Additionally, the use of the term can include from any minimal decrease to absolute abolishment of a physiological process or effect.

As used herein with respect to conditions of the eye, “disease” refers to any condition of the eye that can result in diminished, abnormal, or lost ocular function. This includes congenital disorders, pathogenic disorders, and injury arising from physical, chemical, or other trauma. This also includes trauma or other disturbance arising from procedures conducted on the eye and intended to address such conditions.

As used herein, “subject” refers to an animal, such as a mammal, that may benefit from the administration of a bioconjugate compound of the present disclosure, including formulations or compositions that include the compound.

As used herein, the term “taxane” generally refers to a class of diterpenes produced by the plants of the genus Taxus (yews). This term also includes those taxanes that have been artificially synthesized. For example, this term includes paclitaxel and docetaxel, and derivatives thereof.

As used herein, the term “cobalamin” refers to an organocobalt complex having the essential structure shown below:

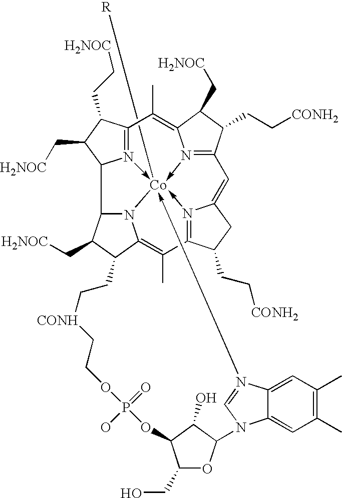

as well as derivatives of this structure in which R may be —CH₃ (methylcobalamin), —CN (cyanocobalamin), —OH (hydroxocobalamin), —C₁₀H₁₂N₅O₃ (deoxyadenosylcobalamin), or synthetic complexes that include a corrin ring and are recognized by cobalamin transport proteins, receptors, and enzymes. The term also encompasses inclusion of substituent groups on the corrin ring that do not eliminate its binding to transport proteins. The term “organocobalt complex” refers to an organic complex containing a cobalt atom having bound thereto 4-5 calcogens as part of a multiple unsaturated heterocyclic ring system, particularly any such complex that includes a corrin ring.

The organocobalt molecule cobalamin is an essential biomolecule with a stable metal-carbon bond. Among other things, cobalamin plays a role in the folate-dependent synthesis of thymidine, an essential building block of DNA. Because cobalamin is a large molecule, cellular uptake of cobalamin is achieved by receptor-mediated endocytosis. The density of receptors in a cell may be modulated in accordance with the cell's need for cobalamin at a given time. For example, a cell may upregulate its expression of cobalamin receptors during periods of high demand for cobalamin. One such time is when the cell replicates its DNA in preparation for mitosis or meiosis. One result of this facultative upregulation is that cobalamin uptake will be higher in cell populations undergoing rapid proliferation than in slower-growing cell populations. This non-uniform uptake profile makes it possible to target delivery of a bioactive agent to high-demand cell populations by linking the agent to cobalamin.

Cobalamin is the most chemically complex of the vitamins. The core structure of the cobalamin molecule is a corrin ring including four pyrrole subunits, two of which are directly connected with the remainder connected through a methylene group. Each pyrrole has a proprionamide substituent that extends radially from the ring. At the center of the ring is a cobalt atom in an octahedral environment that is coordinated to the four corrin ring nitrogens, as well as the nitrogen of a dimethylbenzimidazole group. The sixth coordination partner can vary as previously discussed; represented by R in formula I. Six propionamide groups extend from the outer edge of the ring, while a seventh links the dimethylbenzimidazole group to the ring through a phosphate group and a ribose group.

The term “vitamin B₁₂” or “B₁₂” has been generally used in two different ways in the art. In a broad sense, it has been used interchangeably with four common cobalamins: cyanocobalamin, hydroxocobalamin, methylcobalamin, and adenosylcobalamin. In a more specific way, this term refers to only one of these forms, cyanocobalamin, which is the principal B₁₂ form used for foods and in nutritional supplements. For the purposes of this invention, this term includes cyanocobalamin, hydroxocobalamin, methylcobalamin, and adenosylcobalamin, unless the context dictates otherwise.

As used herein, the term “bioconjugate” refers to a molecule containing a taxane covalently bonded to cobalamin, e.g., directly to the cobalt atom or by some other linkage mechanism.

Exemplary of the bioconjugate function is the ability to solubilize the taxane upon conjugation. As such, the present bioconjugates can have water solubility allowing for direct dissolution of the bioconjugate in water without the need for solubilization excipients. For example, a taxane can be solubilized with CREMOPHOR®; however, such a solution is toxic, which limits its therapeutic effectiveness and administration. However, the present bioconjugates allow solubilization of taxanes in water, or other aqueous solutions, without the need for further excipients, which decreases toxicity and allows for intra-ocular delivery.

Additionally, in one embodiment, the bioconjugate function can serve as a targeted delivery system where the agent or compound to be delivered may be conjugated or otherwise attached to cobalamin without affecting the cobalamin's ability to bind to the appropriate receptor(s). Therefore, it is often the case that the receptor-binding domain(s) of the cobalamin are not modified. Likewise, for successful targeted delivery, the agent or compound can be released from the cobalamin in a therapeutically effective form and at the right location. Some event, substance, or condition can be present in the targeted location that will cause the agent to separate from the carrier. Successful methods of drug targeting can involve agent-cobalamin linkages that are sensitive to particular conditions or processes that are prevalent in the target location.

As used herein, the term “covalent linkage” or “covalent bond” refers to an atom or molecule which covalently or coordinate covalently binds together two components. With regard to the present disclosure, a covalent linkage is intended to include atoms and molecules which can be used to covalently bind a taxane to cobalamin, such as through the central cobalt atom in one embodiment. Though not excluded, in one embodiment, the covalent linkage does not prevent the binding of cobalamin to its transport proteins, either by sterically hindering interaction between cobalamin and the protein, or by altering the binding domain of cobalamin in such a way as to render it conformationally incompatible with the protein. Likewise, the covalent linkage should not act in these ways to significantly prevent the binding of the cobalamin-transport protein complex with cobalamin receptors.

As used herein, the term “angiogenesis” or “angiogenic” refers to a physiological process involving the growth of new blood vessels. The growth of new blood vessels is an important natural process occurring in the body, both in health and in disease. In regards to certain eye diseases, the term “anti-angiogenic” refers to those compounds or agents that inhibit the growth of new blood vessels, effectively cutting off the existing blood supply of the disease(s). For example, such anti-angiogenic compounds include, but are not limited to, bevacizumab, suramin, sunitinib, thalidomide, tamoxifen, vatalinib, cilenigtide, celecoxib, erlotinib, lenalidomide, ranibizumab, pegaptanib, sorafenib, and mixtures thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 micron to about 5 microns” should be interpreted to include not only the explicitly recited values of about 1 micron to about 5 microns, but also include individual values and sub-ranges within the indicated range. Thus, included in this numerical range are individual values such as 2, 3.5, and 4 and sub-ranges such as 1-3, 2-4, and 3-5, etc. This same principle applies to ranges reciting only one numerical value. Furthermore, such an interpretation should apply regardless of the breadth of the range or the characteristics being described.

In accordance with these definitions, the present invention provides methods of treating eye diseases by administering a composition to a subject where the composition includes a taxane or derivative covalently bound to cobalamin. Alternatively, a method of treating an eye disease can comprise administering a taxane compound to a subject to treat the eye disease, wherein the taxane compound has a water solubility of at least 50 mg/ml. It is noted that when discussing a cobalamin-taxane bioconjugate or taxane compound or a method of administering such a composition, each of these discussions can be considered applicable to other embodiments describe herein, whether or not they are explicitly discussed in the context of that embodiment. Thus, for example, in discussing taxanes bioconjugates or taxane compounds, the details of the methods can be used interchangeably.

In one embodiment, the bioconjugate can comprise a taxane covalently bonded to a cobalamin. In another embodiment, the taxane can be covalently bonded to a central cobalt atom of the cobalamin, and in another embodiment, the bioconjugate can be present as a solubilized compound in an aqueous solution. The step of administering can be accomplished by various methods as are known in the art.

In one embodiment, the step of administering can be by intra-ocular administration or delivery. In another embodiment, the step of administering can be by oral administration or delivery. In yet another embodiment, the step of administering can be by parenteral administration or delivery. In still yet another embodiment, the step of administering can be by topical delivery to the tissue site, or by dermal or mucosal administration or delivery.

The methods of the present invention can be used to treat eye diseases in general, and in one embodiment, eye diseases that can benefit from anti-angiogenic activity. As such, the eye disease can be selected from the group consisting of age-related macular degeneration, proliferative diabetic retinopathy, non-proliferative diabetic retinopathy, retinopathy of prematurity, corneal graft rejection, neovascular glaucoma, rubeosis, pterygia, abnormal blood vessel growth of the eye, uveitis, dry-eye syndrome, post-surgical inflammation and infection of the anterior and posterior segments, angle-closure glaucoma, open-angle glaucoma, post-surgical glaucoma procedures, exopthalmos, scleritis, episcleritis, Grave's disease, pseudotumor of the orbit, tumors of the orbit, orbital cellulitis, blepharitis, intraocular tumors, retinal fibrosis, vitreous substitute and vitreous replacement, iris neovascularization from cataract surgery, macular edema in central retinal vein occlusion, cellular transplantation (as in retinal pigment cell transplantation), cystoid macular edema, pseudophakic cystoid macular edema, diabetic macular edema, pre-phthisical ocular hypotomy, proliferative vitreoretinopathy, extensive exudative retinal detachment (Coat's disease), diabetic retinal edema, diffuse diabetic macular edema, ischemic opthalmopathy, pars plana vitrectomy for proliferative diabetic retinopathy, pars plana vitrectomy for proliferative vitreoretinopathy, sympathetic ophthalmia, intermediate uveitis, chronic uveitis, retrolental fibroplasia, fibroproliferative eye diseases, acquired and hereditary ocular conditions such as Tay-Sach's disease, Niemann-Pick's disease, cystinosis, corneal dystrophies, and combinations thereof.

In one embodiment, the present bioconjugates can treat age related macular degeneration (AMD). Specifically, AMD general can be described in two forms: dry and wet. Dry is most common and does not have neovascularization. However, dry AMD can lead to wet AMD. Wet AMD has neovascularization which is the development of abnormal leaky blood vessels in the macular of the eye. This can result in blindness and/or very impaired vision. Wet AMD is an angiogenic process, i.e., it is the development of new blood vessels that are weak and leaky. These occur in the macula and as a result, can also lead to bleeding in the eyes from the vessels leaking blood. As such, the present bioconjugates can be used for the treatment of AMD, as a result of their anti-angiogenic benefits, as further described herein. Additionally, in another embodiment, the present bioconjugates can treat diabetic retinopathy (both non-proliferative and proliferative) as such diseases are known to have abnormal blood vessel growth.

The present eye diseases can benefit from administration of the present bioconjugates, e.g., B₁₂-paclitaxel, since such bioconjugates are water soluble allowing for direct solubilization in water, or other aqueous solutions, without the need for toxic solubilizing excipients, e.g., CREMOPHOR®. Additionally, the bioconjugates can be nontoxic in the eye at doses up to 85 μg/2 μL.

Generally, attaching the taxane to the cobalt atom of cobalamin more closely approximates the binding arrangement seen in stable, biologically active forms of cobalamin, such as adenosylcobalamin. It has been recognized that the attachment of a taxane to the cobalt atom of a cobalamin can significantly increase the water solubility of the taxane as a cobalamin-taxane bioconjugate. Thus, such an arrangement can be beneficial for treating eye disease, though other forms of such bioconjugates can also be used when solubility is not the objective, e.g., emulsions, microemulsions, liposomes, etc.

Generally, taxanes are insoluble in water. For example, paclitaxel has a water solubility of less than 0.004 mg/ml. However, when conjugated to a cobalt atom of a cobalamin, as shown in the following structure and described herein, a cobalamin-paclitaxel bioconjugate can have water solubility of over 100 mg/ml, though lesser degrees of solubility with certain molecules can also be effective for treatment as well. For example, in one embodiment, a cobalamin-taxane bioconjugate can have a water solubility of at least 0.5 mg/ml. In another embodiment, a cobalamin-taxane bioconjugate can have a water solubility of at least 10 mg/ml. In yet another embodiment, the water solubility can be at least 50 mg/ml. In still yet another embodiment, the water solubility can be at least 100 mg/ml. In one embodiment, at least 80% of the bioconjugate can be dissolved in an aqueous solution prior to administration. It is noted that the cobalamin-taxane bioconjugates provided herein can be orally administered to a subject or can be delivered directly to the eye, or by some other effective administration route. In one embodiment, paclitaxel can be covalently bonded to the cobalt atom of a hydroxocobalamin. Specifically, the cobalamin-taxane bioconjugate can be a cobalamin-paclitaxel bioconjugate having the following structure:

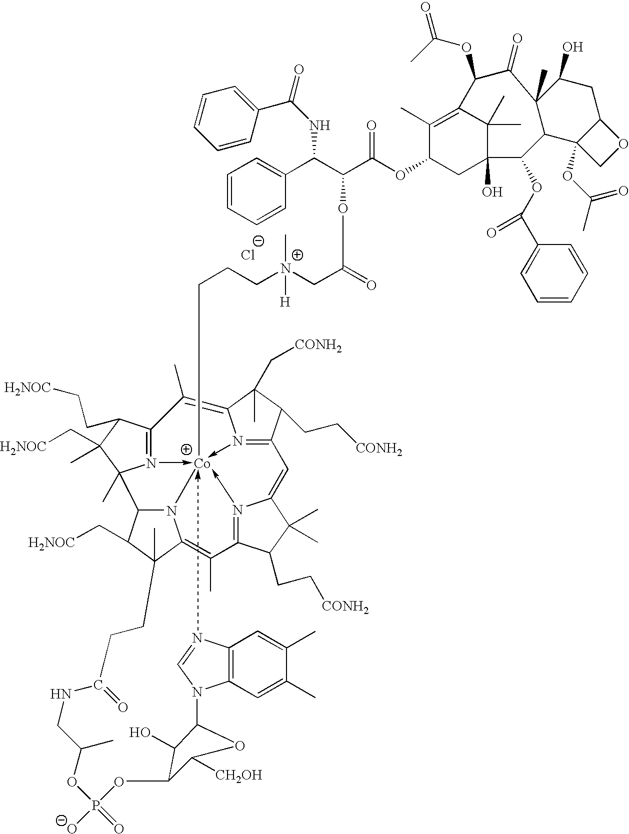

Alternatively, the cobalamin-taxane bioconjugate can be a cobalamin-docetaxel bioconjugate having the following structure:

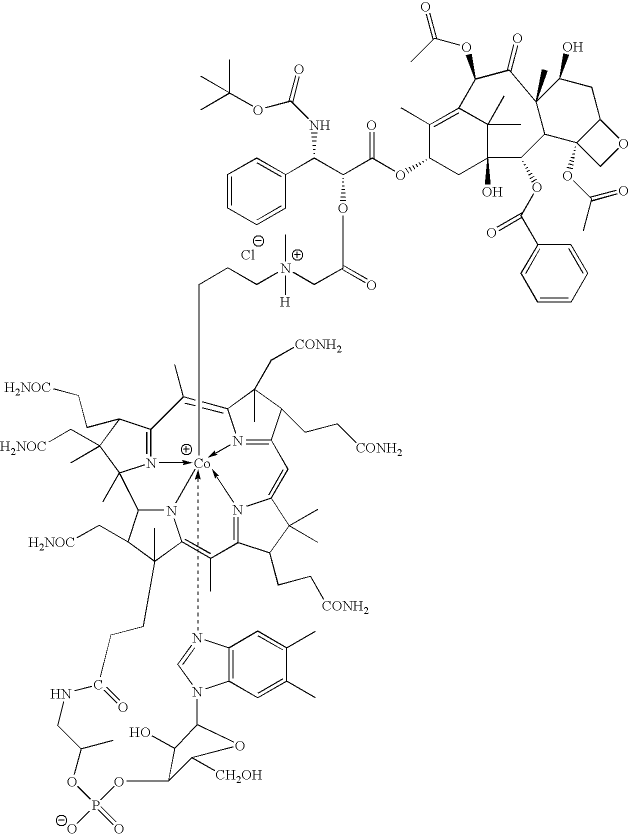

In each of the two above structures as well as in other similar embodiments, it is understood that although the Cl⁻ counter ion is shown, other similar pharmaceutically acceptable counter ions can alternatively be used.

The cobalamin-taxane bioconjugates can have a water solubility several orders of magnitude higher than unconjugated taxanes. In one embodiment, the cobalamin-taxane bioconjugate can have at least a 10-fold increase in water solubility compared to the unconjugated taxane. In another embodiment, the increase can be at least 100-fold. In yet another embodiment, the increase can be at least 1000-fold.

Additionally, it has been recognized that the cobalamin-taxane bioconjugates disclosed herein can have increased bioavailability in a subject. Bioavailability of a compound can be dependent on P-Glycoprotein (P-gp), an ATP-dependent drug pump, which can transport a broad range of hydrophobic compounds out of a cell. This can lead to the phenomenon of multi-drug resistance. Expression of P-gp can be quite variable in humans. Generally, the highest levels can be found in the apical membranes of the blood-brain/testes barrier, intestines, liver, and kidney. Over-expression in patients can undermine treatment as the drug is pumped out via this pump. P-gp can also affect the penetration of the drug to solid tumors or other maladies. P-gp has been shown to affect the ability of taxanes, such as paclitaxel or docetaxel, to enter the cells and become bioavailable. Therefore, the bioconjugates of the present invention can be structurally different as to bypass the P-gp pathway leading to increased bioavailability of the bioconjugate. Additionally, cobalamin bioconjugates can use a facultative transport mechanism, which would also bypass the P-gp pathway leading to increased bioavailability.

The present disclosure also relates to solubilization and drug delivery of taxanes and their derivatives for the treatment of the eye via a cobalamin-taxane bioconjugate, e.g., oral, parenteral, topical, ocular, etc. In addition, it is noted that there may be an inherent targeting effect via the cobalamin molecule. When introduced into the bloodstream or gastrointestinal tract of a subject, such a bioconjugate can take advantage of existing systems for absorption, transport, and binding of cobalamin. In this way, the taxane can be transported to cells that bear receptors for cobalamin and be taken up by those cells. As noted above, some cells or cell populations in a given subject can utilize cobalamin more heavily at a given time than other cells; consequently expression of cobalamin receptors is upregulated in such cells at those times. Thus, when the bioconjugate is administered to a subject, more of the taxane can be taken up by these cells than by other cells. Thus, the present invention provides a method for concentrating a taxane to sites where cells are utilizing cobalamin heavily. Increased demand for cobalamin is associated with, among other things, rapid cellular proliferation. Therefore, the present invention can be used to concentrate taxanes in neoplastic cells in a subject suffering from a proliferative disease.

The taxane can be covalently bonded to the cobalt atom directly or through a covalent linkage. The linkage serves as a connection between the cobalamin and the taxane, and can serve to achieve a desired distance between these two components, while preferably not negatively affecting the binding of the bioconjugate to proteins involved in cobalamin metabolism. In particular, the linkage can include an ester linkage. Alternatively or additionally, the linkage can include a quaternary amine. In another alternative embodiment, the linkage could be a hydrazone linkage. The bioconjugate of the present invention can also include a linkage comprising a polymethylene, carbonate, ether, acetal, or any combination of these units.

Though specific structures and discussions are provided above, it is noted that in a more general embodiment, the cobalamin-taxane bioconjugate can be linked as follows:

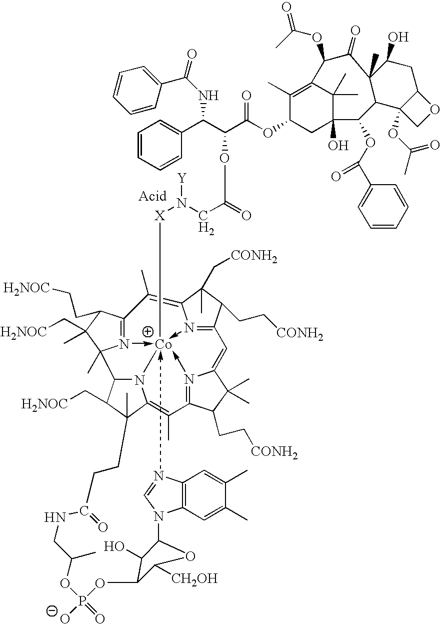

where Y is any alkyl containing 1 to 4 carbons; and X is an optionally substituted, saturated, branched, or linear, C₁₋₅₀ alkylene, cycloalkylene or aromatic group, optionally with one or more carbons within the chain being replaced with, N, O or S, and wherein the optional substituents are selected from carbonyl, carboxy, hydroxyl, amino and other groups. The “Acid” can be any organic or inorganic acid, preferably having the ability to form pharmaceutically acceptable salts. Other linkages that will serve the functions described above will be known to those having skill in the art, and are encompassed by the present invention.

Such a linkage can serve as a target for an enzyme that will cleave the linkage, releasing the taxane from the cobalamin. Such an enzyme can be present in the subject's bloodstream and thereby release the taxane into the general circulation, or it can be localized specifically to a site or cell type that is the intended target for delivery of the taxane. Alternatively, the linkage can be of a type that will cleave or degrade when exposed to a certain environment or, particularly, a characteristic of that environment such as a certain pH range or range of temperatures. The linkage can be of a “self-destructing” type, i.e. it will be consumed in the process of cleavage, so that said cleavage will yield only the original cobalamin and the taxane molecules absent any remaining large sections of the linkage. Those having skill in the art will recognize other release mechanisms derived from various linkages that can be used in accordance with the present invention.

Again, though specific compounds are shown by way of example, it is understood that many different combinations of taxanes and cobalamin can be prepared in accordance with embodiments of the present disclosure. For example, the taxane for use can be selected from the group consisting of paclitaxel and docetaxel, derivatives thereof, and mixtures thereof. In one embodiment, the taxane can be paclitaxel. In another embodiment, the taxane can be docetaxel. The cobalamin can be selected from the group consisting of cyanocobalamin including anilide, ethylamide, proprionamide, monocarboxylic, dicarboxylic, and tricarboxylic acid derivatives thereof; hydroxycobalamin including anilide, ethylamide, proprionamide, monocarboxylic, dicarboxylic, and tricarboxylic acid derivatives thereof; methylcobalamin including anilide, ethylamide, proprionamide, monocarboxylic, dicarboxylic, and tricarboxylic acid derivatives thereof; adenosylcobalamin including anilide, ethylamide, proprionamide, monocarboxylic, dicarboxylic, and tricarboxylic acid derivatives thereof; aquocobalamin; cyanocobalamin carbanalide; desdimethyl cobalamin; monoethylamide cobalamin; methlyamide cobalamin; 5′-deoxyadenosylcobalamin; cobamamide derivatives; chlorocobalamin; sulfitocobalamin; nitrocobalamin; thiocyanatocobalamin; benzimidazole derivatives including 5,6-dichlorobenzimidazole, 5-hydroxybenzimidazole, trimethylbenzimidazole, as well as adenosylcyanocobalamin; cobalamin lactone; cobalamin lactam; 5-o-methylbenzylcobalamin; derivatives thereof; mixtures thereof; and analogues thereof wherein the cobalt is replaced by another metal. In one embodiment, the cobalamin can be one of the vitamin B₁₂ types of cobalamin, and in one specific embodiment, hydroxocobalamin.

The compounds of the present invention can be administered as pharmaceutical compositions in treating various eye diseases. Notwithstanding the ability to solubilize taxanes without the need for solubilizing excipients and/or additives, such a composition can further comprise one or more excipients, including binders, fillers, lubricants, disintegrants, flavoring agents, coloring agents, sweeteners, thickeners, coatings, and combinations thereof. The composition of the present invention can be formulated into a number of dosage forms including syrups, elixirs, solutions, suspensions, emulsions, capsules, tablets, lozenges, and suppositories. Differing administration regimens will call for different dosage forms, depending on factors such as the subject's age, medical condition, level of need for treatment, as well as the desired time course of therapeutic effect. Those having skill in the art will recognize that various classes of excipients can each provide different characteristics to a pharmaceutical composition and that they can be combined in certain ways in accordance with the present invention to achieve an appropriate dosage form. The present invention provides compounds that can be administered to a subject intra-ocularly, orally, dermally, or parenterally.

One aspect of the present invention is that administering the bioconjugate can be more effective in treating an eye disease than administering the taxane and the cobalamin as separate molecules. In light of the fact that taxanes alone can provide anti-angiogenic effects, the present invention provides cobalamin-taxane bioconjugates as anti-angiogenic compounds for treating various eye diseases. The amount of taxane to cobalamin can generally be equal, e.g., the taxane to cobalamin molar ratio can about 1:1. However, the composition can have an excess of cobalamin or taxane that is not covalently bonded. In one embodiment, a composition can have a cobalamin to cobalamin-taxane bioconjugate molar ratio from about 1.2:1 to about 10:1. Additionally, the bioconjugate can further include additional anti-angiogenic compounds. Such additional anti-angiogenic compounds include, but are not limited to, bevacizumab, suramin, sunitinib, thalidomide, tamoxifen, vatalinib, cilenigtide, celecoxib, erlotinib, lenalidomide, ranibizumab, pegaptanib, sorafenib, and mixtures thereof.

As previously discussed, the bioconjugates of the present invention are readily soluble in water and can be administered to a subject having various eye diseases. As such, the administering can be therapeutically effective while providing low serum levels in the patient, enabling effective treatments having no or very little toxicity. Specifically, the serum levels can be less than 0.01 ng/ml. In another embodiment, the serum levels can be less than 0.001 ng/ml. The taxane of the bioconjugate can be administered at, or equivalent to, about 0.001 μg/day to about 10 μg/day.

As cobalamin receptors are highly upregulated in rapidly proliferating cells as dividing cells require cobalamin for thymidine synthesis in DNA replication. This makes cobalamin a useful vehicle to preferentially deliver drugs to proliferating cells. In one embodiment, administering the bioconjugates of the present invention can be used to achieve serum levels in a subject of about 0.1 ng/ml to about 20,000 ng/ml. Further, the taxanes of the cobalamin-taxane bioconjugates of the present invention can be administered at about 1 mg/kg/day to about 10 mg/kg/day. In one embodiment, the rate can be about 2 mg/kg/day to about 6 mg/kg/day.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

### EXAMPLES

The following provides examples of taxane bioconjugates in accordance with the compositions and methods previously disclosed. Additionally, some of the examples include studies performed showing the effects of oral taxanes on animals in accordance with embodiments of the present invention.

### Example 1

### Preparation of Cobalamin-Paclitaxel Bioconjugate

A cobalamin-paclitaxel bioconjugate was prepared using the following reaction schematic:

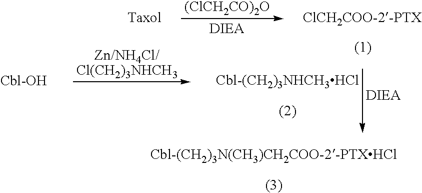

### Abbreviations:

Cbl-: β-substituted cobalamin
PTX: paclitaxel
DIEA: diisopropylethylamine

A Waters Alliance® 2695 HPLC system and a Waters Alliance® 2996 PDA detector are used for analysis of the bioconjugate. A 50 mM H₃PO₄ solution (adjusted to pH 3.0 with ammonia; buffer A) and acetonitrile/water (9:1; buffer B) are used as aqueous and organic eluents, respectively, unless stated otherwise. Waters Delta-Pak® C₁₈ 15 μm 100 Å 3.9 mm×300 mm column (P/N WAT011797) and a 1 ml/min flow rate are also used. Mass spectra is acquired on PE-Sciex API 2000 Mass Spectrometer. The intermediate products, labeled (1)-(3) in the schematic, are each prepared as follows:

### Preparation of (1) CICH₂COO-2′-PTX

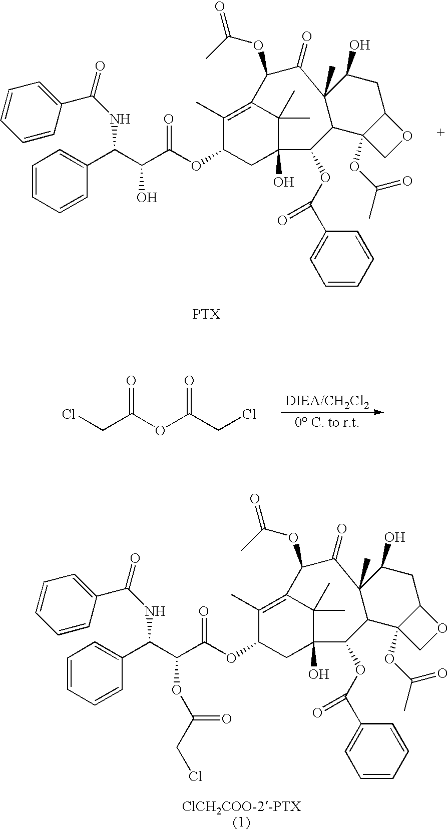

To a stirred solution containing paclitaxel (1.074 g, 1.258 mmol) in CH₂Cl₂ (7 ml) is added 2-chloroacetic anhydride (0.236 g, 1.376 mmol) and DIEA (0.26 ml, 1.376 mmol) consequently at 0° C. The reaction is slowly warmed up to room temperature. After 24 hrs, the reaction mixture is concentrated purified by flash chromatography (silica gel, 0-80% ethyl acetate in hexane) and 0.987 g (84.33%) of white solid is obtained.

### Preparation of (2) Cbl-(CH₂)₃NHCH₃.HCl

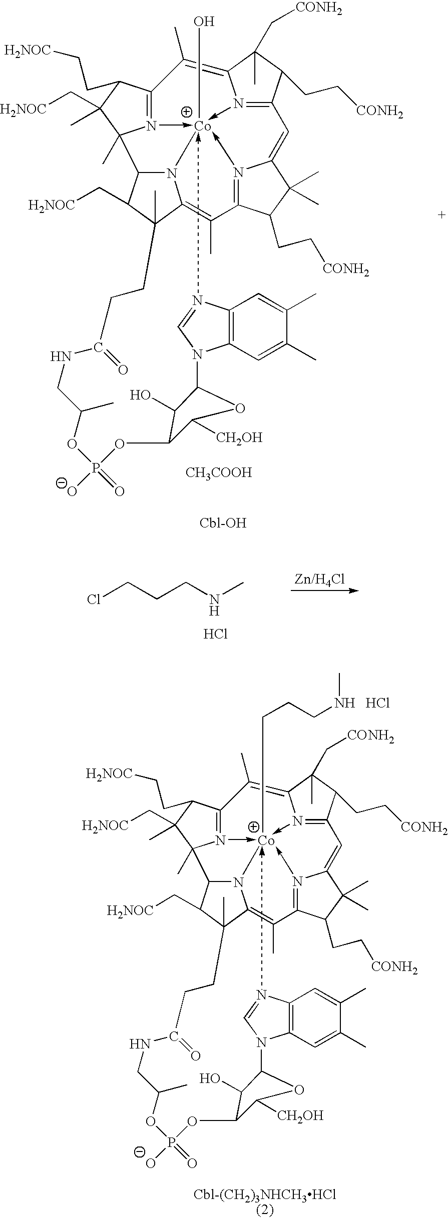

Hydroxocobalamin acetate (0.5 g, 0.355 mmol) is dissolved in DI H₂O (25 ml), and N-methyl-3-chloropropylamine (0.108 g, 0.751 mmol) and NH₄Cl (0.195 mg, 3.63 mmol) is added to the solution. The solution is degassed by bubbling with N₂ for 30 min. Then, 0.238 g Zn dust (3.63 mmol; <10 micron) is added in one portion. All the starting material is consumed after the reaction is stirred under N₂ for 3.5 h. The reaction mixture is then filtered with Whatman No. 42 filter paper to remove Zn. The filtrate is loaded on a Waters C18 Sep-Pak® cartridge (10 g of C₁₈ sorbant) that is pre-washed by washing with 60 ml of methanol followed with 100 ml of water. All salts are removed from the cartridge with 100 ml of water and the product is eluted with CH₃OH—H₂O (9:1) and concentrated to dry. The residue is resuspended in 4 ml of methanol and precipitated in 100 ml of 1:1 (V/V) CH₂Cl₂/anhydrous Et₂O. The red solid is filtered and washed with acetone (20 ml) and ether (20 ml), affording 0.482 g (yield 94.6%, purity 98%) of product.

### Preparation of (3) Cbl-(CH₂)₃N(CH₃)CH₂COO-2′-P7X

A solution of compound (1) (0.743 g, 0.799 mmol, 1.0 eq), compound (2) (1.976 g, 1.374 mmol, 1.72 eq), and DIEA (0.24 ml, 1.374 mmol, 1.72 eq) in DMSO (48 ml) is stirred at room temperature for 3 days. HPLC is employed to confirm consumption of compound 1. The reaction mixture is added to stirring CH₂Cl₂/ether (1:2, 450 ml). The resulting precipitate is collected, washed with CH₂Cl₂ (20 ml×3) and ether (20 ml×3), and air-dried. The crude product is diluted with 0.01 N HCl (200 ml) and applied to a C₁₈ reverse phase 43 g column which is pre-washed sequentially with 7 volumes of methanol and water. The column is first washed with water (50 ml) and eluted with 5-40% B in buffer A (200 ml each with 5% increment). The fractions are checked for purity by HPLC. The desired fractions are combined, diluted with one volume of water, and adsorbed onto a Waters C18 Sep-Pak® cartridge (10 g, P/N WAT043350, pre-washed sequentially with 3 volumes of methanol and water). The product is washed with water (20 ml×3), 0.01 M HCl (20 ml×3), water (20 ml×3) and eluted off the cartridge with 9:1 acetonitrile/water (50 ml). The organic solvent is removed with a rotary evaporator. The residue is dissolved in 0.01 N hydrochloride solution (40 ml, with the aid of a few drops of 0.1 N hydrochloride solution), filtered by 0.45 μm NYLON membrane filter, and lyophilized. 780 mg (41.9%) of red powder is obtained. ES(+)-MS: 1148.9 [(M+H)²⁺], 1329.9 (Cbl⁺), 665.7 [(Cbl+H)²⁺], 971.6 [(Cbl−359)⁺], 359.1 (fragment from the breakdown of C—OP(O) bond).

The resultant compound has the following structure:

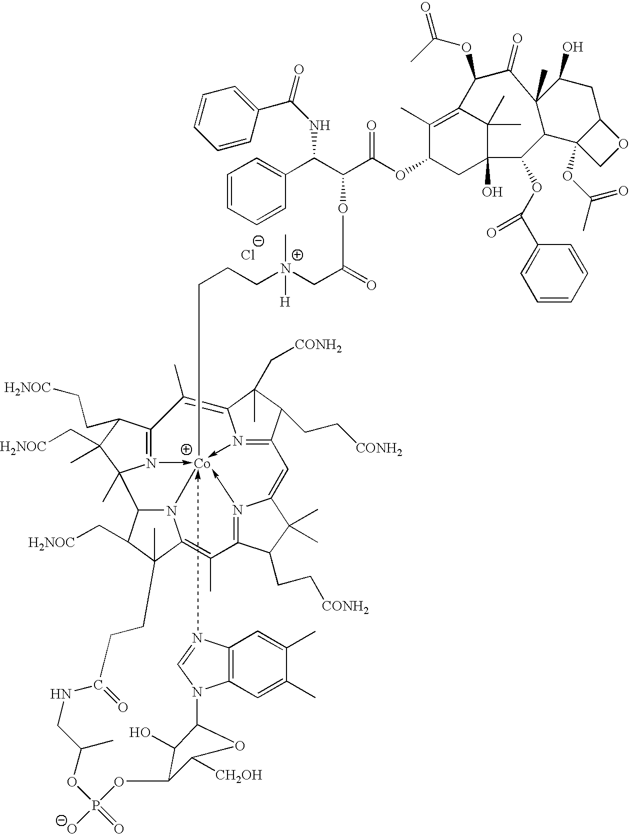

### Example 2

### Preparation of Cobalamin-Docetaxel Bioconjugate

Similar procedures are followed as outlined in Example 1, but with docetaxel as the principal taxane, resulting in the following structure:

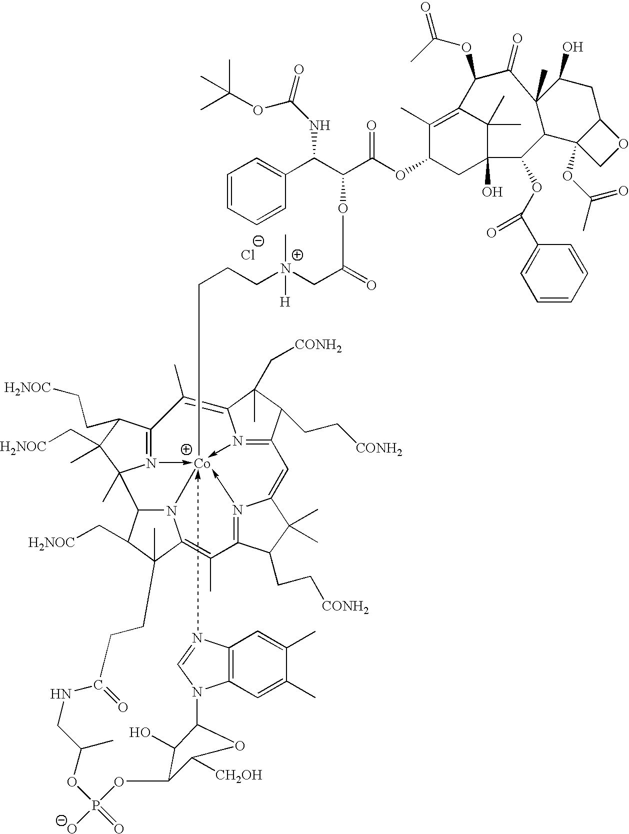

### Example 3

### Cobalamin-Paclitaxel Bioconjugate Dose Study

A group of 6 mice are administered various dosages of the cobalamin-paclitaxel bioconjugate prepared in accordance with Example 1 over a 28-day period. The effects on counts of viable circulating endothelial cell precursors and white blood cells are measured after 28 days. Corresponding amounts of the cobalamin-paclitaxel bioconjugate, viable circulating endothelial cell precursors (CEPs), and white blood cells are presented in the Table 1:

TABLE 1

| Amount of paclitaxel | | |
|---|---|---|
| (in mg/kg) delivered as | Viable CEPs per | White blood cells |
| a cobalamin-paclitaxel | microliter of | per 10⁴ peripheral |
| bioconjugate | peripheral blood | blood cells |
| 0.0 (control) | 1.5 | 6800 |
| 30 | 1.2 | 8100 |
| 6 | 0.9 | 6700 |
| 3 | 0.4 | 7000 |
| 2 | 0.25 | 6700 |
| 1.5 | 0.4 | 6700 |

As can be seen from Table 1, administration of the cobalamin-paclitaxel bioconjugate has an anti-angiogenic effect (marked decrease in viable CEPs) at each dose. However, the most effective dose is not proportional to the amount of paclitaxel administered. In fact, the most effective dose in this particular study is about 2 mg/kg. Furthermore, the absence of a decrease in the white blood cell count shows that such a dosage is less toxic to the mouse (no neutropenia).

### Example 4

### Anti-Angiogenic Efficacy of Cobalamin-Paclitaxel Bioconjugate by Matrigel Plug Perfusion Assay

A Matrigel® plug perfusion in vivo assay is performed to determine the anti-angiogenic efficiacy of the cobalamin-paclitaxel bioconjugate (Cob-Pac) of Example 1. The assay uses Matrigel®, a gelatinous protein mixture secreted by mouse tumor cells (BD Biosciences, San Jose, Calif.), to duplicate tissue environments. Matrigel® is liquid at room temperature, but when injected into the animal, forms a solid plug. If a growth vessel stimulant such as basic fibroblast growth factor (bFGF) is mixed with the Matrigel®, the bFGF stimulates the formation of new blood vessel in the plug, which can be monitored in the animal via fluorescence techniques. In the current study, Matrigel® is injected either alone or with bFGF subcutaneously into mice. Then, as indicated in Table 2, groups of mice are either treated by oral gavage with the cobalamin-paclitaxel conjugate or in the last group with the mouse anti-VEGF receptor antibody, DC101. The results are shown in Table 2:

TABLE 2

| | Matrigel ® Plug/Plasma |
|---|---|
| Assay | Fluorescence Ratio |
| Water with Matrigel ® | 0.00050 |
| Water with Matrigel ® and bFGF | 0.00125 |
| Cob-Pac with Matrigel ® and bFGF | 0.00110 |
| (30 mg/kg expressed in paclitaxel units) | |
| Cob-Pac with Matrigel ® and bFGF | 0.00050 |
| (6 mg/kg expressed in paclitaxel units) | |
| Cob-Pac with Matrigel ® and bFGF | 0.00070 |
| (2 mg/kg expressed in paclitaxel units) | |
| DC101 with Matrigel ® and bFGF | 0.00072 |
| (800 μg/kg) | |

Such results indicate that the addition of bFGF stimulates the growth of blood vessels on the Matrigel® assay as indicated by the fluorescence ratio in the Matrigel® plus bFGF. The addition of cobalamin-paclitaxel bioconjugate inhibits the growth of new blood vessels in each instance shown. However, the greatest effect is seen at the 2 mg/kg (expressed in paclitaxel units) and 6 mg/kg (expressed in paclitaxel units) doses. The cobalamin-paclitaxel bioconjugate can provide better performance than that of DC101, an effective rodent specific anti-angiogenic compound that is well known in the art.

### Example 5

### Choroidal Neovascularization Model

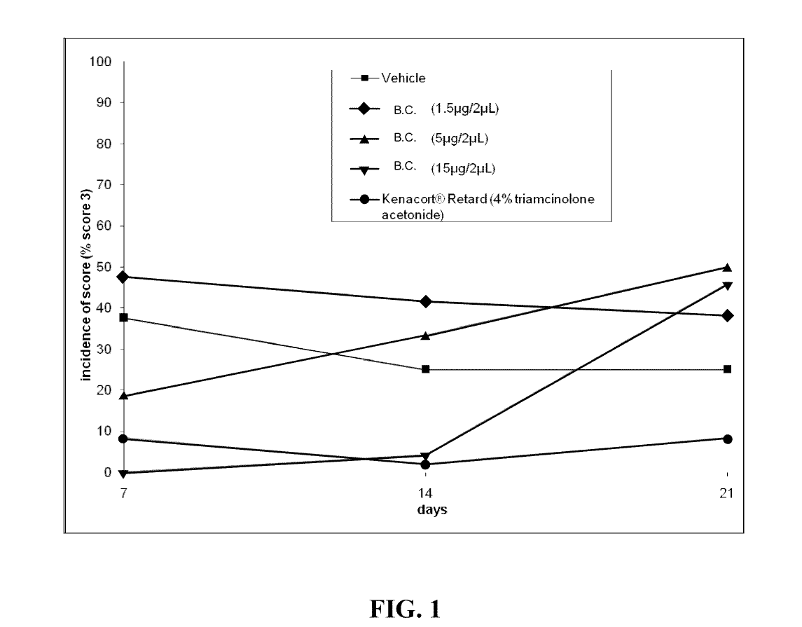

Groups of 8 rats/dosage group or vehicle are neovascularized by laser burns on the eye. Afterwards, the eye is immediately treated with a cobalamin-paclitaxel bioconjugate prepared in accordance with Example 1 at a dose of 1.5 μg/2 μL, 5.0 μg/2 μL, and 15 μg/2 μL (indicated as B.C. in FIG. 1). The treatment regimen also includes a vehicle and Kenacort Retard® (4% triamcinolone acetonide), as a positive control. Each treatment is scored at 7, 14, and 21 days post-treatment by infusing the eye with fluorescein and scoring the leakage using angiography. A score of 0 indicates no leakage while a score of 3 indicates severe leakage. The results of the test are shown in FIG. 1 as the percentage of mice scoring 3.

As can be seen in FIG. 1, after 7 days, the present bioconjugate in the intermediate and high doses show anti-angiogenic results. After 14 days, the high dose still provides anti-angiogenic benefit. Such results show that the compound of Example 1 can be effective for preventing new BV growth in the eye.

### Example 6

### Lesion Study

In another study, flat mount evaluation of the eyes can be carried out at the end of the study because angiography may not provide a full evaluation of the effect of the drug. At the end of the study, the eyes are removed, histologically processed and all lesions can be seen (including those that can not be detected by angiography). Such an evaluation can be used as a better measure of the choroidal neovascularization model.

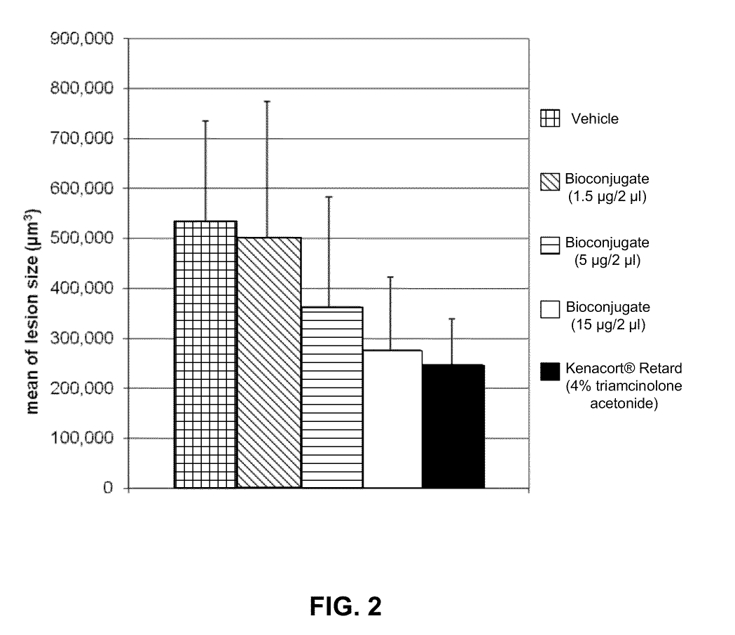

Groups of 8 rats/dosage group are neovascularized by laser burns, followed by immediate treatment of the eye with a cobalamin-paclitaxel bioconjugate prepared in accordance with Example 1. Dosages used are 1.5 μg/2 μL, 5.0 μg/2 μL, and 15 μg/2 μL. The treatments also include a vehicle and Kenacort Retard® (4% triamcinolone acetonide). After 21 days, the eyes are removed, histologically processed, and the lesion size scored in μm³. FIG. 2 shows the results of the treatments.

As illustrated in FIG. 2, the number of blood vessel lesions decrease in a concentration dependent manner after treatment using the B₁₂-paclitaxel bioconjugate of Example 1, indicating dose dependent inhibition of blood vessel growth. As such, the present study provides a more detailed analysis than the angiography results from Example 5. The present study demonstrates that both the high and medium concentrations of B₁₂-paclitaxel can be efficacious in inhibiting new blood vessel growth.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A method of treating an eye disease, comprising administering a bioconjugate to a subject to treat the eye disease, wherein the bioconjugate comprises a taxane covalently bonded to a cobalamin.

2. The method of claim 1, wherein the taxane is covalently bonded to a cobalt atom of the cobalamin.

3. The method of claim 1, wherein at least 80% of the bioconjugate is dissolved in an aqueous solution prior to administration.

4. The method of claim 1, wherein the bioconjugate has a water solubility of at least 50 mg/ml.

5. The method of claim 1, wherein the bioconjugate has a water solubility of at least 100 mg/ml.

6. The method of claim 1, wherein the step of administering achieves serum levels of about 0.1 ng/ml to about 20,000 ng/ml of the taxane in the subject.

7. The method of claim 1, wherein the taxane portion of the bioconjugate is administered at about 1 mg/kg/day to about 10 mg/kg/day.

8. The method of claim 1, wherein the taxane portion of the bioconjugate is administered at about 2 mg/kg/day to about 6 mg/kg/day.

9. The method of claim 1, wherein the eye disease is selected from the group consisting of age-related macular degeneration, proliferative diabetic retinopathy, non-proliferative diabetic retinopathy, retinopathy of prematurity, corneal graft rejection, neovascular glaucoma, rubeosis, pterygia, abnormal blood vessel growth of the eye, uveitis, dry-eye syndrome, post-surgical inflammation and infection of the anterior and posterior segments, angle-closure glaucoma, open-angle glaucoma, post-surgical glaucoma procedures, exopthalmos, scleritis, episcleritis, Grave's disease, pseudotumor of the orbit, tumors of the orbit, orbital cellulitis, blepharitis, intraocular tumors, retinal fibrosis, vitreous substitute and vitreous replacement, iris neovascularization from cataract surgery, macular edema in central retinal vein occlusion, cellular transplantation, cystoid macular edema, pseudophakic cystoid macular edema, diabetic macular edema, pre-phthisical ocular hypotomy, proliferative vitreoretinopathy, extensive exudative retinal detachment (Coat's disease), diabetic retinal edema, diffuse diabetic macular edema, ischemic opthalmopathy, pars plana vitrectomy for proliferative diabetic retinopathy, pars plana vitrectomy for proliferative vitreoretinopathy, sympathetic ophthalmia, intermediate uveitis, chronic uveitis, retrolental fibroplasia, fibroproliferative eye diseases, acquired and hereditary ocular conditions such as Tay-Sach's disease, Niemann-Pick's disease, cystinosis, corneal dystrophies, and combinations thereof.

10. The method of claim 1, wherein the taxane includes a member selected from the group consisting of paclitaxel and docetaxel, derivatives thereof, and mixtures thereof.

11. The method of claim 1, wherein the taxane is paclitaxel.

12. The method of claim 1, wherein the taxane is docetaxel.

13. The method of claim 1, wherein the cobalamin includes a member selected from the group consisting of cyanocobalamin including anilide, ethylamide, proprionamide, monocarboxylic, dicarboxylic, or tricarboxylic acid derivatives thereof; hydroxocobalamin including anilide, ethylamide, proprionamide, monocarboxylic, dicarboxylic, or tricarboxylic acid derivatives thereof; methylcobalamin including anilide, ethylamide, proprionamide, monocarboxylic, dicarboxylic, or tricarboxylic acid derivatives thereof; adenosylcobalamin including anilide, ethylamide, proprionamide, monocarboxylic, dicarboxylic, or tricarboxylic acid derivatives thereof; aquocobalamin; cyanocobalamin carbanalide; desdimethyl cobalamin; monoethylamide cobalamin; methlyamide cobalamin; 5′-deoxyadenosylcobalamin; cobamamide derivatives; chlorocobalamin; sulfitocobalamin; nitrocobalamin; thiocyanatocobalamin; benzimidazole derivatives including 5,6-dichlorobenzimidazole, 5-hydroxybenzimidazole, trimethylbenzimidazole, or adenosylcyanocobalamin; cobalamin lactone; cobalamin lactam; 5-o-methylbenzylcobalamin; derivatives thereof; mixtures thereof; and analogues thereof.

14. The method of claim 1, wherein the cobalamin is a hydroxocobalamin.

15. The method of claim 1, wherein the cobalamin is vitamin B₁₂.

16. The method of claim 1, wherein the administering is by ocular delivery.

17. The method of claim 1, wherein the administering is by oral delivery or by parenteral delivery.

18. The method of claim 1, wherein the administering is by topical tissue or dermal delivery.

19. The method of claim 1, wherein the bioconjugate has a taxane to cobalamin molar ratio of about 1:1.

20. The method of claim 1, wherein the bioconjugate is present in a composition with an excess of cobalamin that is not covalently bonded to the taxane.

21. The method of claim 20, wherein the composition has a cobalamin to bioconjugate molar ratio from about 1.2:1 to about 10:1.

22. The method of claim 1, wherein the taxane is covalently bonded to the cobalamin through an ester linkage.

23. The method of claim 1, wherein the taxane is covalently bonded to the cobalamin through a quaternary amine.

24. The method of claim 1, wherein the taxane covalently bonded to the cobalamin is paclitaxel covalently bonded to a cobalt atom of a hydroxocobalamin.

25. The method of claim 1, wherein the bioconjugate is a cobalamin-paclitaxel bioconjugate, comprising the structure:

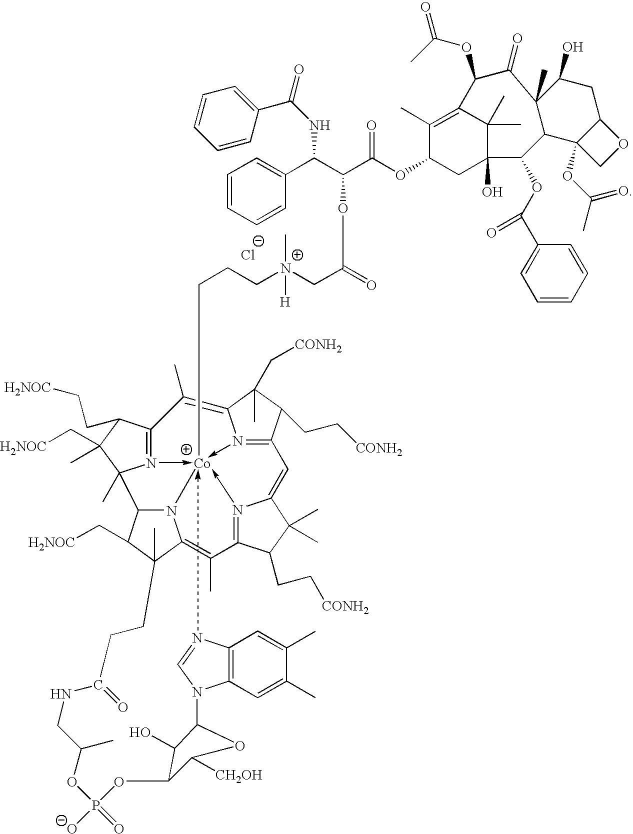

26. The method of claim 25, wherein the water solubility of the cobalamin-paclitaxel bioconjugate is at least 50 mg/ml.

27. The method of claim 25, wherein the water solubility of the cobalamin-paclitaxel bioconjugate is at least 100 mg/ml.

28. The method of claim 1, wherein the taxane covalently bonded to the cobalamin is docetaxel covalently bonded to a cobalt atom of cobalamin.

29. The method of claim 1, wherein the bioconjugate is a cobalamin-docetaxel bioconjugate, comprising the structure:

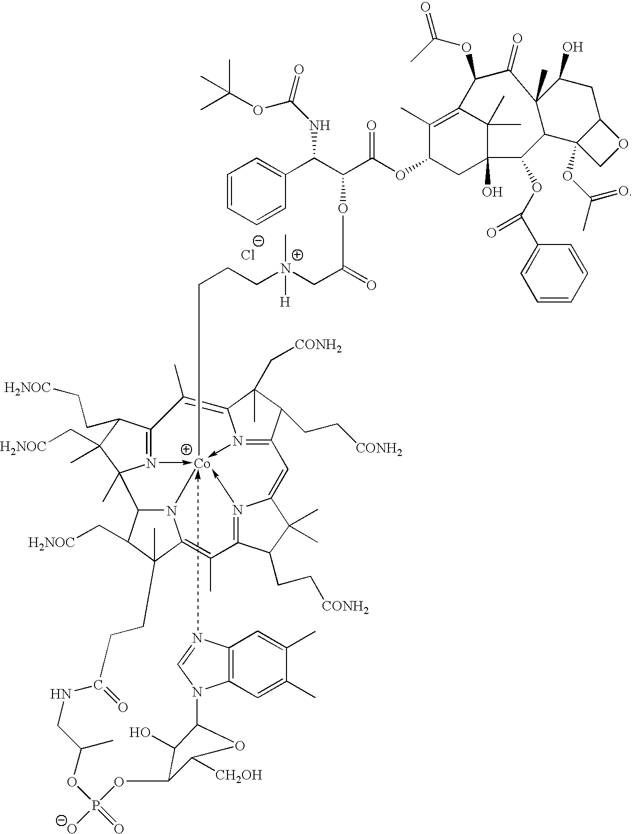

30. The method of claim 29, wherein the water solubility of the cobalamin-docetaxel bioconjugate is at least 50 mg/ml.

31. The method of claim 29, wherein the water solubility of the cobalamin-docetaxel bioconjugate is at least 100 mg/ml.

32. A method of treating an eye disease, comprising administering a taxane compound to a subject to treat the eye disease, wherein the taxane compound has a water solubility of at least 50 mg/ml.

33. The method of claim 32, wherein the taxane compound is a bioconjugate comprising a taxane covalently bonded to a cobalt atom of a cobalamin.

34. The method of claim 33, wherein the bioconjugate has a taxane to cobalamin molar ratio of about 1:1.

35. The method of claim 33, wherein the bioconjugate is present in a composition with an excess of cobalamin that is not covalently bonded to the taxane.

36. The method of claim 35, wherein the composition has a cobalamin to bioconjugate molar ratio from about 1.2:1 to about 10:1.

37. The method of claim 33, wherein the taxane is covalently bonded to the cobalamin through an ester linkage.

38. The method of claim 33, wherein the taxane is covalently bonded to the cobalamin through a quaternary amine.

39. The method of claim 33, wherein the bioconjugate comprises paclitaxel covalently bonded to a cobalt atom of a hydroxocobalamin.

40. The method of claim 33, wherein the cobalamin includes a member selected from the group consisting of cyanocobalamin including anilide, ethylamide, proprionamide, monocarboxylic, dicarboxylic, or tricarboxylic acid derivatives thereof; hydroxocobalamin including anilide, ethylamide, proprionamide, monocarboxylic, dicarboxylic, or tricarboxylic acid derivatives thereof; methylcobalamin including anilide, ethylamide, proprionamide, monocarboxylic, dicarboxylic, or tricarboxylic acid derivatives thereof; adenosylcobalamin including anilide, ethylamide, proprionamide, monocarboxylic, dicarboxylic, or tricarboxylic acid derivatives thereof; aquocobalamin; cyanocobalamin carbanalide; desdimethyl cobalamin; monoethylamide cobalamin; methlyamide cobalamin; 5′-deoxyadenosylcobalamin; cobamamide derivatives; chlorocobalamin; sulfitocobalamin; nitrocobalamin; thiocyanatocobalamin; benzimidazole derivatives including 5,6-dichlorobenzimidazole, 5-hydroxybenzimidazole, trimethylbenzimidazole, or adenosylcyanocobalamin; cobalamin lactone; cobalamin lactam; 5-o-methylbenzylcobalamin; derivatives thereof; mixtures thereof; and analogues thereof.

41. The method of claim 33, wherein the cobalamin is a hydroxocobalamin.

42. The method of claim 33, wherein the cobalamin is vitamin B₁₂.

43. The method of claim 32, wherein at least 80% of the taxane compound is dissolved in an aqueous solution prior to administration.

44. The method of claim 32, wherein the taxane compound has a water solubility of at least 100 mg/ml.

45. The method of claim 32, wherein the step of administering achieves serum levels of about 0.1 ng/ml to about 20,000 ng/mi of the taxane in the subject.

46. The method of claim 32, wherein the taxane portion of the taxane compound is administered at about 1 mg/kg/day to about 10 mg/kg/day.

47. The method of claim 32, wherein the eye disease is selected from the group consisting of age-related macular degeneration, proliferative diabetic retinopathy, non-proliferative diabetic retinopathy, retinopathy of prematurity, corneal graft rejection, neovascular glaucoma, rubeosis, pterygia, abnormal blood vessel growth of the eye, uveitis, dry-eye syndrome, post-surgical inflammation and infection of the anterior and posterior segments, angle-closure glaucoma, open-angle glaucoma, post-surgical glaucoma procedures, exopthalmos, scleritis, episcleritis, Grave's disease, pseudotumor of the orbit, tumors of the orbit, orbital cellulitis, blepharitis, intraocular tumors, retinal fibrosis, vitreous substitute and vitreous replacement, iris neovascularization from cataract surgery, macular edema in central retinal vein occlusion, cellular transplantation, cystoid macular edema, pseudophakic cystoid macular edema, diabetic macular edema, pre-phthisical ocular hypotomy, proliferative vitreoretinopathy, extensive exudative retinal detachment (Coat's disease), diabetic retinal edema, diffuse diabetic macular edema, ischemic opthalmopathy, pars plana vitrectomy for proliferative diabetic retinopathy, pars plana vitrectomy for proliferative vitreoretinopathy, sympathetic ophthalmia, intermediate uveitis, chronic uveitis, retrolental fibroplasia, fibroproliferative eye diseases, acquired and hereditary ocular conditions such as Tay-Sach's disease, Niemann-Pick's disease, cystinosis, corneal dystrophies, and combinations thereof.

48. The method of claim 32, wherein the taxane compound includes a member selected from the group consisting of paclitaxel and docetaxel, derivatives thereof, and mixtures thereof.

49. The method of claim 32, wherein the administering is by ocular delivery.

50. The method of claim 32, wherein the administering is by oral delivery or by parenteral delivery.
